(19) United States
(12) Patent Application Publication
(10) Pub. No.: US 2015/0290053 A1
(43) Pub. Date: Oct. 15, 2015

(54) PROTECTIVE CAST COVER

(71) Applicant: Keep It Dry, Inc., Sewickley, PA (US)

(72) Inventors: Roger Loughney, Sewickley, PA (US); John Weld, Morgantown, WV (US)

(21) Appl. No.: 14/682,498

(22) Filed: Apr. 9, 2015

Related U.S. Application Data
(60) Provisional application No. 61/977,389, filed on Apr. 9, 2014.

(51) Int. Cl. A61F 15/00

(52) U.S. Cl. CPC A61F 15/004

(57) ABSTRACT

Disclosed is a method and apparatus to protect a limb having a wound or condition requiring dryness from moisture. The method of applying a protective covering for a wound or condition requiring dryness comprising the steps of positioning a waterproof polyester fabric sock around an appendage having a gasket secured to an open end of the sock for sealing the sock to an appendage.

### PRIORITY CLAIM

In accordance with 37 C.F.R. 1.76, a claim of priority is included in an Application Data Sheet filed concurrently herewith. Accordingly, the present invention claims priority to U.S. Provisional Patent Application No. 61/977,389, entitled “PROTECTIVE CAST COVER”, filed Apr. 9, 2014. The contents of which the above referenced application is incorporated herein by reference.

### FIELD OF THE INVENTION

This invention is directed to the medical field of art using casts to immobilize body parts and in particular, to a waterproof cast cover.

### BACKGROUND OF THE INVENTION

Casts are used for immobilizing a body part, such as a broken bone, in need of stabilization. The most common type of cast is formed from a cotton bandage wrapped with calcined gypsum. The calcined gypsum is mixed with water to form a material, commonly referred to as a plaster cast, which can be formed into most any shape.

Plaster casts are relatively bulky being formed by wrapping the area to be immobilized with a cotton wrap, the wrapping must be smooth so as to eliminate pressure sores. The calcined gypsum is the activated with water and wrapped around the cotton wrap circumferentially. The material initiates an exothermic chemical reaction and begins to dry within minutes. However, plaster casts are susceptible to water damage and the cotton web can act like a sponge and absorb moisture. Degradation of the plaster or saturating the cotton can require the individual wearing the cast to have the cast removed and replaced, adding an expense, inconvenience, and discomfort.

The plaster cast and be mixed with most any synthetic material, such as fiberglass, polyurethane, thermoplastic and the like to reduce the susceptibility to moisture. However, such combinations can lessen the moldability and have adverse reactions with skin. Plaster can be easily molded to fit patient's limbs and support the bone placement more precisely. Should the bone be out of position, once the bone is manipulated back into position, the plaster can be used to help the bone into the proper position. The plaster cast is formed outside the initial layer and is of a mixture that can harden quickly, but has a disadvantage, in that it can dissolve if it gets wet.

If a lower extremity such as the leg is injured, the extremity may include the ankle or foot. Immobilization of the foot creates another problem for conventional footwear cannot be worn. The individual must walk on the cast leading to early degradation of the cast. Leg casts may include walking materials placed along the bottom of the foot which is capable of withstanding wear. However, it still remains that the cast itself is made of plaster, and is susceptible to early degradation should the plaster become wet.

Keeping a cast dry at all times for months at a time can be difficult considering the individual must bath. Further in tropical areas such as Florida or moist areas such as Washington, keeping the cast dry is even more difficult. Further, problems can occur when the cast is worn by a child or an active individual. Even if a cast is covered by clothing, should the individual be exposed to rain, the cast can soften resulting in the degradation of the effectiveness of the cast and possibly affecting recovery time.

An alternative to plaster casts is fiberglass which is known to be lighter and water proof but again need a skin wrap to prevent skin irritation. The fiberglass cast may include a waterproof lining but fiberglass tends to provide a less comfortable fitting and is not routinely used. Further, fiberglass casts are expensive and not readily accepted by insurance companies.

For the above reasons, it is beneficial to cover casts to prevent degradation of the material or saturation of the cotton wrap that surrounds the skin. Various cast covers are known and include U.S. Pat. No. 7,867,179 which discloses a disposable cast cover that is sealed with an elongated piece of high cling plastic stretch film used in the packing industry which is self-clinging to itself. U.S. Pat. No. 4,986,265 discloses a protective cast cover which covers a cast with a protective plastic cover and wraps the end of the protective cover with a neoprene or foam plastic material to form the seal. U.S. Pat. No. 4,911,151 discloses a disposable dressing cover for an arm or a leg provides for a tubular plastic sleeve to fit over the cast and then has one end secured with a strap which is attached by an adhesive. U.S. Pat. No. 5,643,183 discloses a waterproof cover for a cast uses a transparent polyethylene bag and seals the distal ends with pairs of strips of hook and loop material. U.S. Pat. No. 4,036,220 discloses a protective cover and has a flexible collar to form the seal which is attached with hook and loop material. U.S. Pat. No. 2,229,575 discloses a flexible bag covering an artificial limb which is sealed with a sealing band. U.S. Pat. No. 5,342,286 discloses a water impervious covering for extremities and uses an adhesive strip in the sealing of a flexible covering. U.S. Pat. No. 4,363,317 discloses a watertight cast cover which is sealed with a band having overlapping ends forming flaps of hook and loop material. U.S. Pat. No. 4,610,245 discloses a medical protective sleeve for a human limb which has a sealing cuff which is wrapped tightly around the limb. U.S. Pat. No. 3,741,203 discloses a protective covering for an injured limb which forms a seal with wrapping tape. U.S. Pat. No. 5,016,622 discloses an immobilizing orthopedic cast made of an inner water-impermeable protective sleeve next to the skin and a resilient padding layered with a plastic or resin or glass fiber outer immobilizing layer. U.S. Pat. No. 4,523,586 and U.S. Pat. No. 3,324,580 discloses a protective cover for a limb or cast having a sealing band. U.S. Pat. No. 5,592,953 discloses a wound cover is formed with a flexible sleeve sealed at each end. U.S. Pat. No. 6,916,301 discloses a waterproof cover for casts formed as a thin-walled elastomeric water-impervious linear tube made of nitrile.

Thus, what is needed in the industry is an improved cover for casts capable of temporarily sealing the cast from water degradation. Such a cover can also be used protect splints and other wounds stemming from any procedure, medical or not, that requires an area of the upper or lower extremity to be kept dry.

### SUMMARY OF THE INVENTION

An apparatus and method for protecting an area on an upper or lower extremity of a use from moisture. An exemplary model is an apparatus and method for protecting a cast from moisture, however, the present invention can also be used for splints, stitches, abrasions, burns, or other conditions which might afflict an extremity of the user. Solely for illustrative purposes, this application will specifically refer to casts, however, as stated above, the invention can be used for any condition on the extremity which requires the area to be kept free of moisture.

The apparatus is formed from a waterproof covering forming a sock for receipt of the appendage immobilized by the cast. The sock can be sized for children up to extra large adults and includes a gasket that is secured to the sock by weldment or attachment by sewing with a taped seal. The gasket can be made out of latex, or alternatively from a non-latex gasket material such as polyisoporene or isoprene.

The gasket can be further sealed to the appendage by adhesive tape to form a waterproof seal around the cast suitable for bathing. The method of applying the protective covering to a cast comprising the steps of positioning a polyester fabric sock preferably constructed from 70d Taffeta weave sublimation fabric with water proof coating around an appendage. A gasket is secured to an open end of the sock by tape and/or weldment and the gasket can be further sealed to an appendage by adhesive tape creating an impervious water shield around the cast.

Accordingly, an objective of the invention is to provide a cast cover capable of providing waterproof protection of a cast to increase patient comfort and allow the cast wearer the ability to spend time in the rain, shower, bath, or even swim.

A further objective is to provide a cast cover that prevents moisture from reaching the cast lining to inhibit the growth of mildew, bacteria and fungi on the cast lining and thereby reduce itching, rashes and odor caused with a spoiled inner liner. By reducing the continual skin contact with moisture, the chance for maceration and necrosis of the skin is greatly reduced.

Still another objective of the invention is to teach a protective cover that can be adapted for various arm and leg sizes.

A further objective of the invention is to provide a cast cover that is breathable to prevent a buildup of sweat from moistening the inner lining of the cast.

A yet further objective of the invention is to provide a cast cover that is durable and will not easily tear or rip, allowing a user to use the cover throughout the entire time the user wears a cast.

A further objective of the invention is to teach a waterproof cast cover that is machine washable for improved hygiene.

A still further objective of the invention is to provide a cast cover that is also waterproof even when fully submerged in water so that a user can swim without risk that the cast and lining will get wet.

Other objectives and further advantages and benefits associated with this invention will be apparent to those skilled in the art from the description, examples and claims which follow.

### BRIEF DESCRIPTION OF THE DRAWINGS

FIG. 1 is a pictorial illustrating the protective cover placed on an individual's arm and leg;

FIG. 2 is an exploded view of an arm sock and gasket;

FIG. 3 is an exploded view of a four piece leg assembly;

FIG. 4 illustrates manufacturing steps;

FIG. 5 is an illustration of two layer fabric taping;

FIG. 6 is an illustration of gasket taping;

FIG. 7 is an illustration of arm cover sizing;

FIG. 8 is an illustration of leg cover sizing;

FIG. 9 illustrates positioning of the woven label for the arm cover; and

FIG. 10 is an illustration of the woven label for use with the leg protective cover.

### DETAILED DESCRIPTION OF THE INVENTION

Detailed embodiments of the instant invention are disclosed herein, however, it is to be understood that the disclosed embodiments are merely exemplary of the invention, which may be embodied in various forms. Therefore, specific functional and structural details disclosed herein are not to be interpreted as limiting, but merely as a basis for the claims and as a representation basis for teaching one skilled in the art to variously employ the present invention in virtually any appropriately detailed structure.

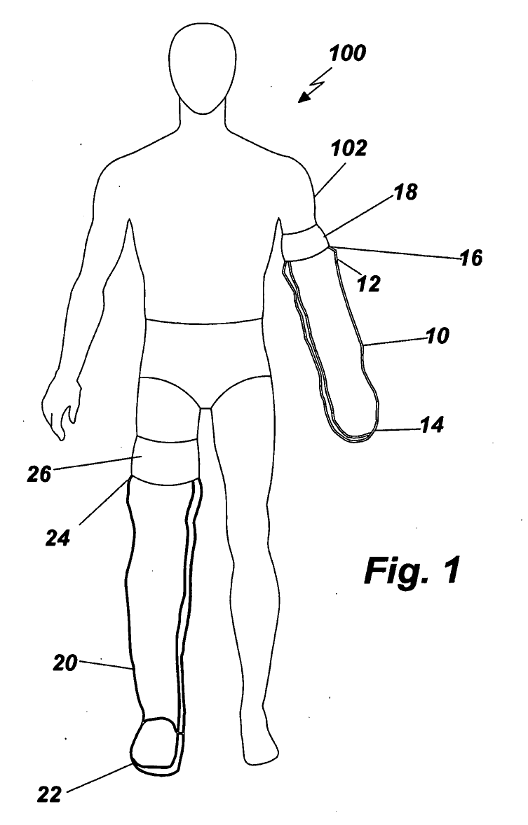

Referring to FIG. 1, set forth is a pictorial of an individual 100 having an arm appendage 102 set in a cast, not shown, for purposes of immobilizing the arm. In this illustration, the cast may extend from the elbow to the wrist for purposes of immobilizing a broken bone. The protective cover of the instant invention consists of a sock 10 formed from a polyester material that is preferably constructed of two pieces, which are sewn together, and the seams taped to form a completely water tight enclosure. The sock has an opening 12 along a proximal end and is sealed along a distal end 14. Sealment of the sock along the distal end encompasses the hand and prevents water from entering the cast along the distal end. A gasket 16 is sewn and seam taped to the waterproof fabric sock 10 along the proximal end 12. The gasket 16 can be made from latex or a non-latex material such as isoprene or polyisoprene.

In one embodiment, the latex gasket is of a flexibility including the ability to stretch with the latex being of surgical grade so as not to cause irritation to the skin. The protective cast cover is slid over the individual's hand, wrist, and forearm by insertion of the hand through the open end along distal end 12. The gasket 16 is sized for either a wrist or bicep attachment, and can be further taped 18 to the skin so as to prevent movement of the protective cover or in instances where the gasket has been over sized. Similarly, a protective leg cover 20 is depicted, consisting of a polyester fabric leg sock which is preferably made from four pieces that are sewn together and seam taped to form a water tight enclosure. The distal end 22 forming coverage of the individual's foot is preferably reinforced with urethane to prevent premature wear of the sock, and allows the individual to walk on the protective cover without early degradation. Similar to the arm cover, a gasket 24 is secured to the leg sock 20 along a proximal end, and is sown to the leg sock and the seams are taped, creating a water tight connection. The thigh gasket, either latex or a non-latex material such as isoprene or polyisoprene, is preferably sized to provide a seal against the individual's thigh, but may include a tapable thigh gasket 26 so as to assure sealment of the gasket against the thigh and prevent unwanted movement.

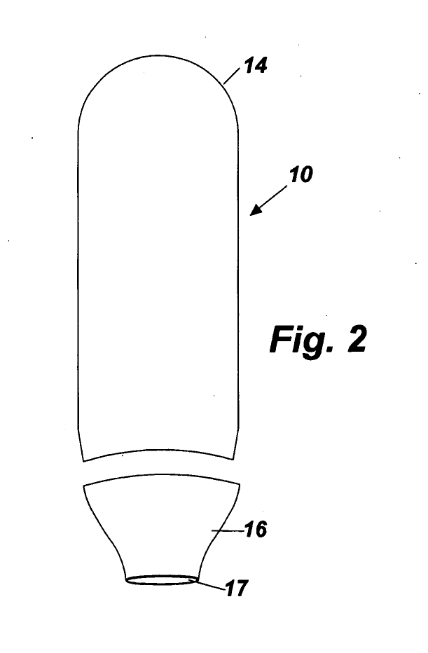

FIG. 2 depicts an exploded view of the arm cast cover 10, having a main section which is preferably made from 70d Taffeta weave sublimation fabric with water proof coating. The arm sock is preferably made from two pieces which are sewn together and sealed with the tape as described later in this specification. The distal end 14 of the sock provides a circumferential seal so as to cover the hand and wrist of the individual. The gasket 16 is sewn and seam taped to the waterproof fabric sock 10, or may be made of a weldable material. It should be noted that the protective arm cast cover can be sized for an individual's wrist, and the dimensions changed accordingly. The gasket 16 includes and open end 17 for receipt of the hand of the individual, and depending upon the size of the protective cover, may receive a hand, wrist, forearm and elbow with the gasket 16 being sized to engage the bicep of the individual.

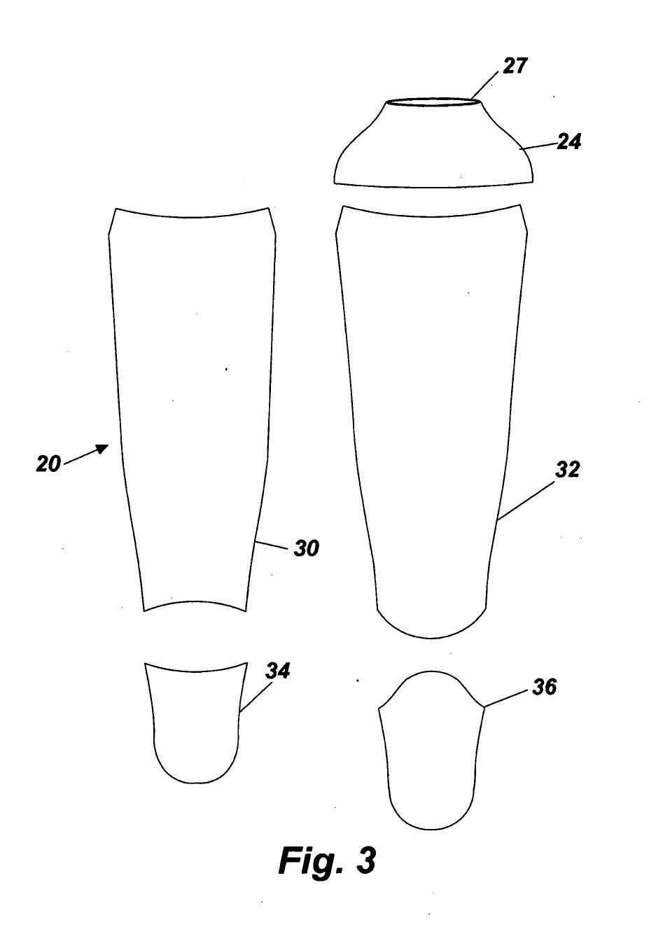

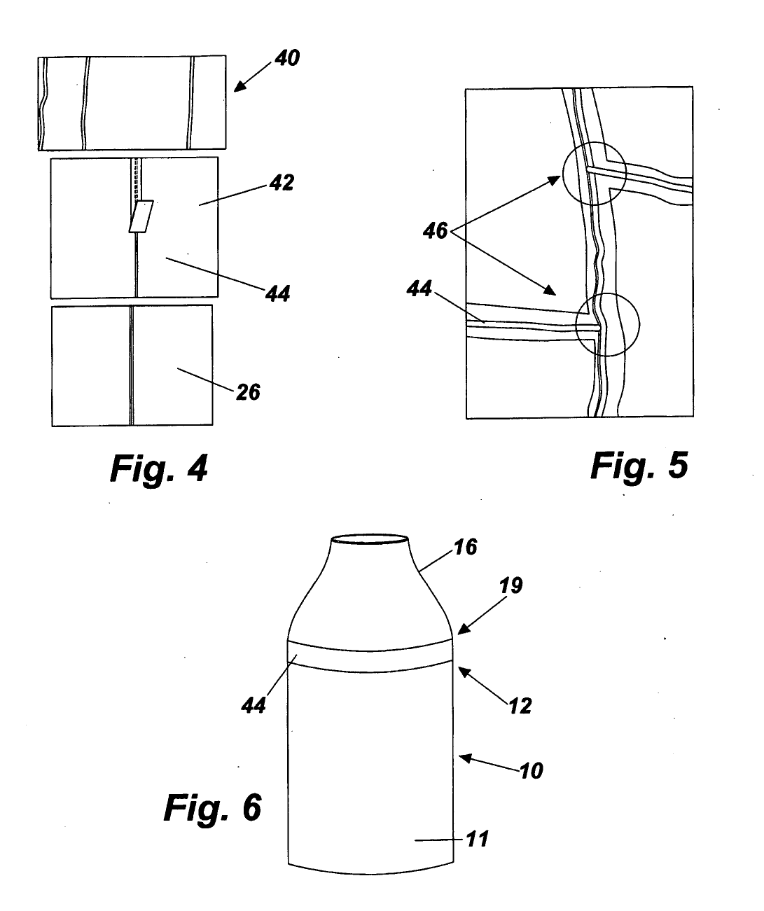

Now referring to FIG. 3, set forth is a protective cast cover for leg 20, which preferably consists of a front leg portion 30, a back leg portion 32, a top front portion for the foot 34, and a bottom foot portion for the foot 36. The sections are attached together by either sewing the sections together with the seams taped to provide a waterproof fabric sock, or the seams can be welded together to create a waterproof sock. Each of the sections, again preferably made from 70d Taffeta weave sublimation fabric with a waterproof coating. The gasket 24 is sized for attachment to an individual's leg and various sizes can be created, as will be explained later in this specification, whereas the opening 27 may be further secured to the thigh by the application of a tapable other material for taping the gasket to the thigh.

FIG. 5 is an illustration illustrating construction wherein all seam allowances 40 are trimmed to approximately 1/16^{th} of an inch. Seam tape 42 is centered on the seam 44, wherein the seam 44 may be welded together as depicted by numeral 46. Illustrated by numeral 48, no top stitching is employed, which provides a smooth surface and prohibits the stitching or seams from being prematurely opened by clothing.

FIG. 6 depicts sock 10 having an inner surface 11, with the distal end 12 attached to the gasket 16, with the seam 44 taped together with Melco t-2000 19.

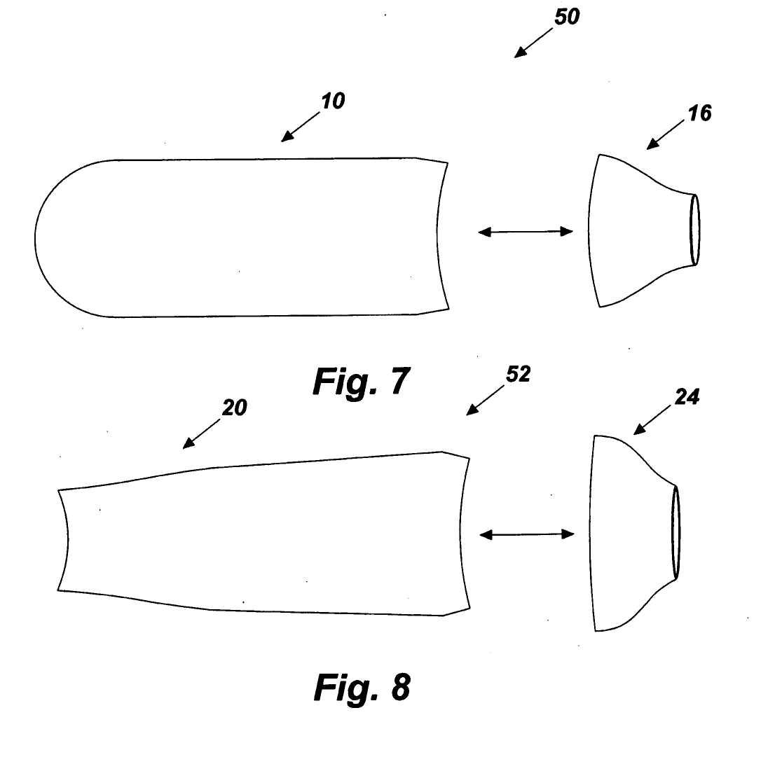

Referring now to FIG. 7, depicted is the arm sock 10 and gasket 16, with a chart 15 depicting arm cover sizing for kids and adults, from small to extra large. Similarly, FIG. 8 depicts the leg sock 20 and gasket 24, with sizing chart 52 depicting kids and adults from small to extra extra large.

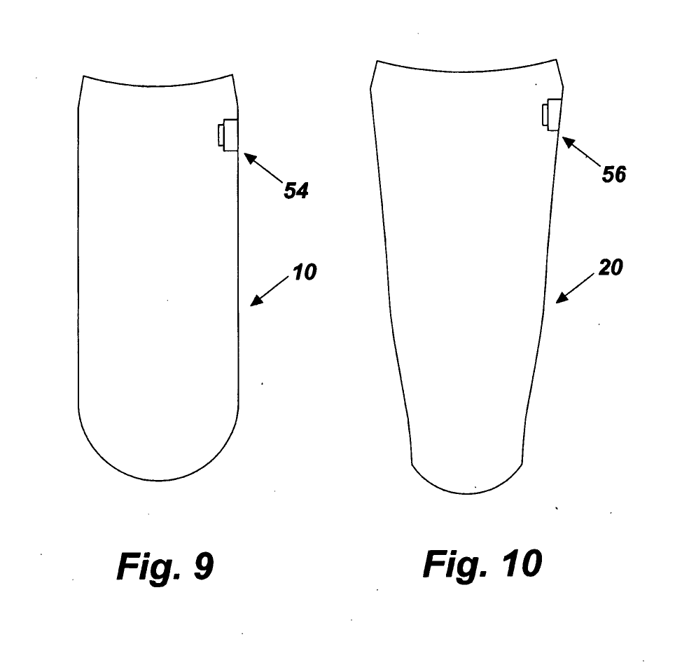

FIG. 9 depicts the leg sock 10 and placement of a woven label 54 for purposes of care, the label art positioned in a location that will not interfere with the protective cover, or inadvertently snag clothing. Similarly, leg sock 20, depicted in FIG. 10, has a label 56, located approximately three inches from the gasket seam to provide public notice as to proper care of the protective cover.

All patents and publications mentioned in this specification are indicative of the levels of those skilled in the art to which the invention pertains. All patents and publications are herein incorporated by reference to the same extent as if each individual publication was specifically and individually indicated to be incorporated by reference. It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A method of applying a protective covering for a wound or condition requiring dryness comprising the steps of:
forming a waterproof polyester sublimation fabric sock constructed and sized for placement around an appendage, said sock having an open end and a closed end;
attaching one continuous edge of a gasket to said open end and sealing said gasket to said open end; and
inserting an individual's appendage afflicted by a wound or condition requiring dryness into said open end of said gasket for placement within said sock, said gasket frictionally engaging the skin of the appendage forming a waterproof seal around the afflicted appendage.

2. The method of applying a protective covering for a wound or condition requiring dryness according to claim 1 wherein said gasket is sealed to said open end with Melco t-2000 tape.

3. The method of applying a protective covering for a wound or condition requiring dryness according to claim 1 wherein said gasket is attached by sewing said gasket to said open end to form a seam that is sealable.

4. The method of applying a protective covering for a wound or condition requiring dryness according to claim 3 wherein said gasket is secured to said open end of said sock by weldment.

5. The method of applying a protective covering for a wound or condition requiring dryness according to claim 1 wherein said waterproof polyester sublimation fabric is a 70d Taffeta weave sublimation fabric with waterproof coating.

6. The method of applying a protective covering for a wound or condition requiring dryness according to claim 1 wherein said fabric sock includes a urethane coating along a distal end of the sock whereby said sock can be used as a walking cast.

7. The method of applying a protective covering for a wound or condition requiring dryness according to claim 1 wherein said gasket is made of latex.

8. The method of applying a protective covering for a wound or condition requiring dryness according to claim 1 wherein said gasket is made of a isoprene.

9. The method of applying a protective covering for a wound or condition requiring dryness according to claim 1 wherein said gasket is made is polyisoprene.

10. The method of applying a protective covering for a wound or condition requiring dryness according to claim 1 wherein said gasket can be further sealed to an appendage by adhesive tape.

11. The method of applying a protective covering for a wound or condition requiring dryness according to claim 1 wherein said waterproof fabric sock is formed by attaching at least two waterproof fabric sheets by sewing and taping the seams to create a waterproof seal.

12. The method of applying a protective covering for a wound or condition requiring dryness according to claim 11 wherein said taping is accomplished by using Melco t-2000.

13. A protective cover for a wound or condition requiring dryness comprising:
a breathable waterproof fabric sock having an open end and a closed end, said sock formed from at least one piece of breathable waterproof polyester sublimation fabric shaped to fit around an appendage of a user;
a gasket having a first open end and a second open end, said first open end attached to form a seam with said sock open end; and
a first securing member attached to said seam, wherein said securing member creates a waterproof seal on said seam;
whereby said protective cover can be placed over an appendage with a wound or condition requiring dryness on it to protect said wound or condition requiring dryness from getting wet when said protective cover is submerged in water.

14. The protective cover for a wound or condition requiring dryness according to claim 13, wherein said sock is formed from at least two pieces of breathable waterproof fabric, said pieces of breathable waterproof polyester sublimation fabric joined together to form a seam, said seam secured by a second securing member, said second securing member forming a waterproof seal on said pieces.

15. The protective cover for a wound or condition requiring dryness according to claim 14 where said first and second securing members are Melco t-2000 tape.

16. The protective cover for a wound or condition requiring dryness according to claim 13, wherein said sock is formed from at least 4 pieces of breathable waterproof polyester sublimation fabric to fit around a human leg, said pieces of breathable waterproof fabric are joined to form at least one seam.

17. The protective cover for a wound or condition requiring dryness according to claim 16 wherein said gasket is polyisoprene.

18. The protective cover for e wound or condition requiring dryness according to claim 16 wherein said at least one seam is formed by sewing and secured by a third securing member, said third securing member forming a waterproof seal on said at least one seam.

19. The protective cover for a wound or condition requiring dryness according to claim 16, further comprising urethane reinforcement on at least a portion of said waterproof polyester sublimation fabric sock.

20. The protective cover for a wound or condition requiring dryness according to claim 14, wherein said breathable waterproof polyester sublimation fabric is 70d Taffetta weave sublimation fabric with waterproof coating.
